(19) United States
(12) Patent Application Publication
(10) Pub. No.: US 2017/0332766 A1
(43) Pub. Date: Nov. 23, 2017

(54) APPLICATOR FOR APPLYING A COSMETIC OR CARE PRODUCT

(71) Applicant: L'OREAL, Paris (FR)

(72) Inventor: Eric CAULIER, Ferrieres (FR)

(73) Assignee: L'OREAL, Paris (FR)

(21) Appl. No.: 15/524,213

(22) PCT Filed: Nov. 3, 2015
(86) PCT No.: PCT/EP2015/075600

§ 371 Date: May 3, 2017

(30) Foreign Application Priority Data
Nov. 3, 2014 (FR) 1460591

(51) Int. Cl. A45D 40/26; B43K 8/00; B43K 23/12

(52) U.S. Cl. CPC A45D 40/262; B43K 23/12; B43K 8/003; A61K 2800/872; A45D 2200/1072

(57) ABSTRACT

Applicator (3) for applying a cosmetic or care product, which extends along a longitudinal axis (X) and has: —an applicator member (6), —a holder (5) for the applicator member, and a handle (10) that defines a gripping surface allowing the user to hold the applicator during the application of the product, the handle being deformable with shape memory at least along a part of its length.

The present invention relates to the application of a cosmetic or care product, in particular a makeup product, to the lips or other human keratin materials, in particular the skin, the nails, the eyelashes or hair. The product may be a makeup product such as an eyeliner, an eyeshadow, a mascara, a lip gloss, a lipstick, a nail varnish or a care product.

The invention relates more particularly but not exclusively to applicators for applying eyeshadow or eyeliner to the eyelids, or nail varnish to the nails, or mascara to the eyelashes, and also to packaging and application devices comprising such applicators.

U.S. Pat. No. 1,950,960 discloses a pencil for writing having a flexible shaft and means for keeping this flexible shaft in a bent position.

In application US 2002/0083953, a makeup brush is retained on a handle by a metal ferrule.

The subject of applications EP 1 504 691 and FR 2 858 529 is a nail varnish applicator, the stem of which has a flexible narrowing with shape memory and a non-flexible lower half.

Application WO 2008/025443 discloses an applicator having a handle folded on itself with portions made of elastomer for giving a degree of flexibility to the application.

U.S. Pat. No. 4,428,388 and U.S. Pat. No. 6,309,125 relate to mascara applicators, the stems of which are flexible and bend when they are released from an enclosing sleeve, with a spring effect.

Finally, applications EP 1 369 055, EP 1 369 059 and WO 2012/168874 relate to applicators having a handle that is articulated via a ball joint or a rotary pin.

Applicators that attempt to make makeup operations easier by trying to improve the way in which the user holds the applicator member have been proposed. In spite of the wide variety of applicators which it has been possible to produce, the makeup operation remains difficult.

Consequently, there is a need to improve this operation without otherwise impairing the quality of the makeup result achieved.

Therefore, according to a first of its aspects, a subject of the invention is an applicator for applying a cosmetic or care product, which extends along a longitudinal axis X and has:
an applicator member,
a holder for the applicator member, and
a handle that defines a gripping surface allowing the user to hold the applicator during the application of the product, the handle being deformable with shape memory at least along a part of its length.

The expression “deformable with shape memory” should be understood as meaning that the handle can be deformed by plastic deformation by the user in order to give it a given shape and that it can preserve this shape while it is being stored or while the applicator is being used to apply a cosmetic or care product, at least until the next intentional deformation by the user. By virtue of its malleability with shape memory, it is possible for the handle not to have an articulation. It may in particular not have a ball joint and a spring.

A “holder” denotes a, preferably rigid, part of the applicator for fitting the applicator member on the handle. The holder may have a stem. The holder may be fixed to a distal end of the handle by any means, for example by snap-fastening, force-fitting, screw-connection, adhesive bonding, overmoulding, crimping, among others.

A “handle” is understood to be the part of the applicator which is held by the user and comes into contact with his/her fingers during normal use of the applicator.

Thus, the applicator according to the invention makes it possible to adapt the shape of the handle to the morphology of the user's hand and/or to his/her personal preferences with regard to the choice of the shape of the handle in order to produce a given makeup effect. The user can easily bring the handle into an ergonomic shape that makes it easier to apply the product and impairs the user as little as possible when he/she is applying the product while looking in a mirror.

The user can thus give the handle a plurality of shapes depending on desired applications, thereby making it possible to vary the ways in which the product is applied and thus the makeup result.

The handle may extend initially without deformation in a rectilinear manner along the longitudinal axis X of the applicator.

The handle may extend along a length L₁ greater than 25 mm, better still greater than 30 or 40 mm, or even greater than 50 or 60 mm. The length L₁ of the handle is measured along the longitudinal axis X of the applicator. The length L₁ may thus be between 25 and 100 mm, better still between 60 and 90 mm.

The handle may have a greatest width l of between 7 and 30 mm, better still between 15 and 25 mm. The greatest width corresponds to the greatest transverse dimension that is measurable along the entire length of the handle. It may be given for example by the diameter of a circle in which the cross section of the handle is inscribed. It may be situated at the distal end of the handle, closest to the applicator member. In one variant embodiment, the width l is substantially constant along the entire length of the handle.

The ratio L₁/l is preferably between 2 and 7, better still between 3 and 6.

The handle may be deformable in a portion that is deformable with shape memory and extends along a length L₂ of between 20 and 100 mm, better still between 30 and 90 mm. The length L₂ is measured along the longitudinal axis X of the applicator. The portion that is deformable with shape memory may extend along the entire length L₁ of the handle or along a shorter length. It may extend along at least half the length L₁ of the handle, or even along at least ⅔, better still at least ¾.

The handle may have a non-deformable portion that is disposed at a proximal end thereof. This non-deformable portion may extend along a length L₃ of between 2 and 50 mm, better still between 15 and 40 mm. The length L₃ is measured along the longitudinal axis X of the applicator. It may extend along less than half the length L₁ of the handle, or even along less than a third, better still along less than a quarter of the length L₁.

The portion that is deformable with shape memory may be closer to the distal end of the handle than to its proximal end.

The handle may have a core that is deformable with shape memory and is made of a first material, in particular a malleable metal material, the core being covered by a sheath made of a second material. The first material may be chosen from the following list, which is not limiting: iron, steel, copper, aluminium.

The second material may be deposited on the first material by overmoulding or by some other method.

It is possible for the first material not to move with respect to the second material. The first material is not constituted by the product to be applied. The second material may be elastically deformable, but the return force that it exerts is insufficient to return the core to its initial shape. Thus, the core can remain in the shape given to it by the user, in spite of the elastically deformable nature of the second material.

It is possible for there not to be adhesion between the first material and the second material, this making it possible to favour the movement of one with respect to the other, and thus to make it easier to deform the handle.

The outer surface of the handle is advantageously elastically deformable, being defined for example by a material that is elastically deformable so as to allow it to be deformed when the user's hand presses on it, so as to soften the contact. The material defining the outer surface of the handle may be soft to the touch. The entire outer surface of the handle may be defined by this material.

In one variant embodiment, the surface of the handle is hard to the touch. The material chosen to define the outer surface of the handle may to this end be chosen to be sufficiently rigid, without otherwise preventing the deformation of the handle with shape memory which is sought according to the invention.

The handle may be configured to be able to be deformed through 360° about the longitudinal axis of the applicator holder.

The force to be applied by the user to start deforming the handle may be between 0.5 and 10 N.

The handle may be configured such that its deformability is limited. Preferably, at its maximum deformation, the inclination of the proximal end of the handle with respect to the longitudinal axis of the applicator holder is greater than 360°. This maximum inclination may remain less than 90° in one embodiment.

The handle may have at least one cross section with a circular shape, or even have a circular cross section along its entire length. In a variant, it may have at least one cross section with a non-circular shape. It may for example have at least one cross section with an elongate shape, for example a lenticular shape, in particular close to its proximal end.

In one variant embodiment, the handle has a cross section with a circular shape close to its distal end and a cross section with an elongate shape, in particular a lenticular shape, close to its proximal end, with a cross section which changes progressively from one to the other along the longitudinal axis of the applicator.

The presence at the proximal end of the handle of a cross section with an elongate shape, in particular a lenticular shape, makes it possible to position the applicator in a satisfactory manner in the user's hand, since the end of the handle can rest more easily on the interdigital web between the thumb and index finger.

The applicator member may be an eyeliner applicator end piece, a brush, in particular a nail varnish brush, or an eyeshadow, lip gloss or lipstick applicator end piece.

The applicator member may be specifically intended for applying eyeliner. It may be a flocked end piece made of elastomer, a brush, a felt, a foam end piece, or an injection-moulded pen which is or is not flocked. It is preferably flexible, having for example a body made of a thermoplastic elastomer. In the case of a felt, it preferably has longitudinally oriented fibres that are bonded together. The product may diffuse by capillary action within the applicator member on account of its porosity. The fibres may be fibres made of thermoplastic material, in particular acrylic polymer, polyester or polyamide.

Along at least a part of its length, the applicator member may have a cross section that is not rotationally symmetric. This makes it possible to draw a more or less fine line depending on the orientation of the applicator member with respect to the direction of movement relative to the eyelid.

The dimensions of the applicator member are such that the maximum thickness of the line of product drawn on the skin is preferably less than or equal to 5 mm, better still less than or equal to 3 mm, when the applicator member is moved perpendicularly to the skin. Depending on the orientation of the applicator member relative to the eyelid, it is possible for example to vary the thickness of the line.

The applicator member may be fixed to the holder by any means, for example force-fitted into a corresponding housing provided at the end of the holder.

Preferably, the applicator member is an applicator tip, which is or is not provided at the end of a spatula or a widened head as described in application FR 2 933 281.

The applicator tip may have an end piece for fixing to the applicator holder which has a diameter greater than the width of the fine line which is drawn by the applicator when the latter is moved in a manner oriented perpendicularly to the skin.

The applicator can serve as a closure cap for a container containing the product to be applied. The holder can be designed to close the container, in particular in a leaktight manner, when it is not being used.

A further subject of the invention is a packaging and application device having a container containing a cosmetic or care product, in particular an eyeliner, to be applied, and an applicator according to the invention, as defined above.

The handle of the applicator can make it possible to ensure the stability of the device when the latter is placed on a flat surface, the handle then preferably being in an extended position. When the handle is in a curved position, it can favour the ergonomics of application.

If need be, the container can have a ball for homogenizing its contents.

The capacity of the container is for example between 1 and 6 ml.

The product may be liquid. The product is for example an eyeliner, a mascara, an eyeshadow, a lip gloss, a lipstick, or a nail varnish.

The eyeliner may comprise an aqueous or organic solvent and a pigment, in particular an iron oxide. The eyeliner contains for example water, a pigment, in particular iron oxide and/or a colorant, and polymers.

The applicator member is supplied with product by being dipped in the container or alternatively the application surface is supplied with product by capillary action through the applicator member, in a similar way to marker pens. In such a variant embodiment, the product, in particular the eyeliner, can be contained in a reservoir disposed inside the handle, the applicator then forming an eyeliner applicator pen. In this case, the applicator member is preferably porous on the inside, with an elongate shape, being supplied with eyeliner by the reservoir.

Another subject of the invention is a makeup method, using an applicator or a device according to the invention, wherein the following steps are implemented:

a) the handle of the applicator is deformed in order to give it a given shape, and

b) the product is applied with the handle of the applicator in the shape it has been given.

A further subject of the invention is a method for making up the eyelid, wherein use is made of an applicator or a device according to the invention to draw a line on the eyelid.

In a variant, the application can be carried out on the entire eyelids, the eyelashes or eyebrows, the skin, the lips or the nails.

The invention may be better understood from reading the following detailed description of non-limiting illustrative embodiments thereof and from examining the appended drawing, in which:

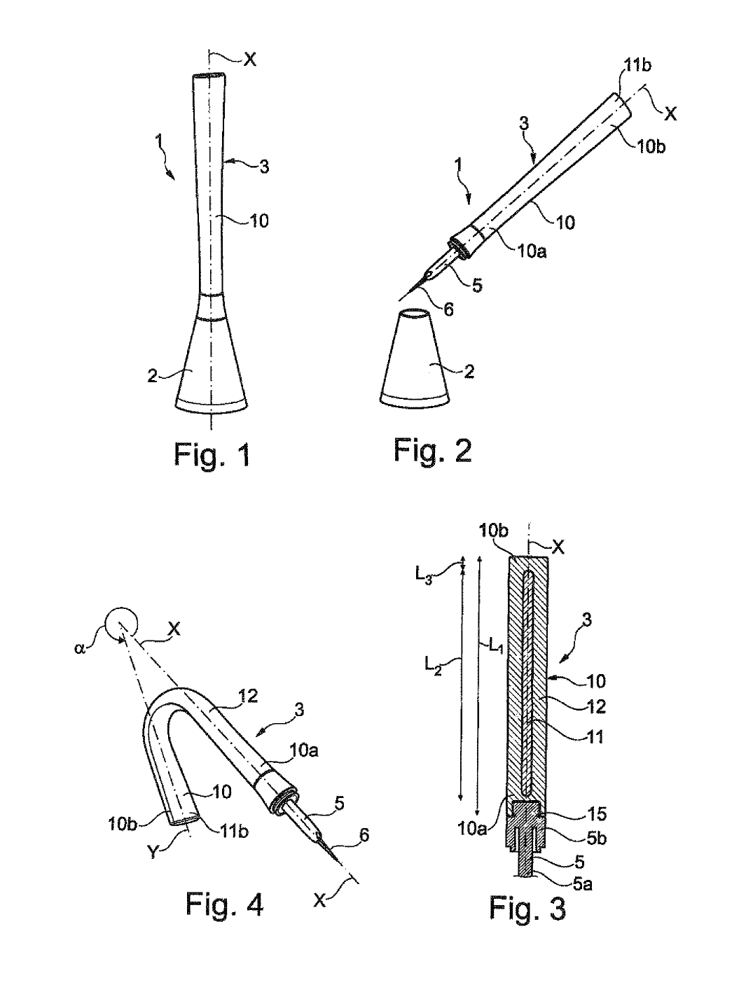

FIGS. 1 and 2 are perspective views, in the closed position and open position, respectively, of an example of a packaging and application device according to the invention,

FIG. 3 is a longitudinal section through said handle,

FIG. 4 is a view of the applicator from FIGS. 1 to 3 after deformation,

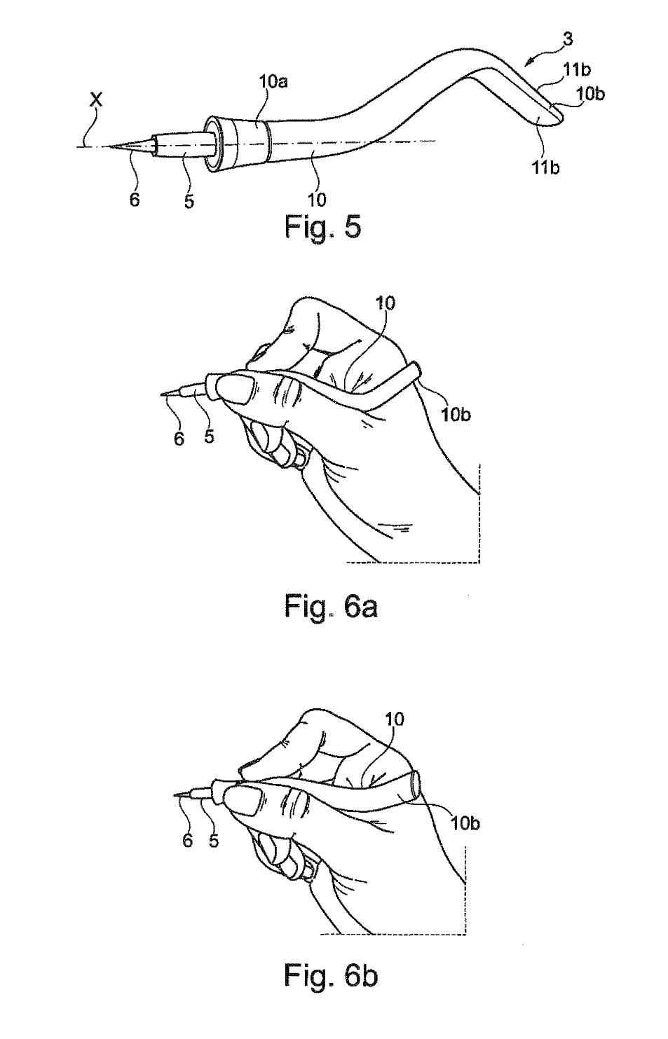

FIG. 5 shows a variant embodiment after deformation,

FIGS. 6a and 6b are perspective views illustrating two possibilities for holding the applicator from FIG. 5 in the hand, and

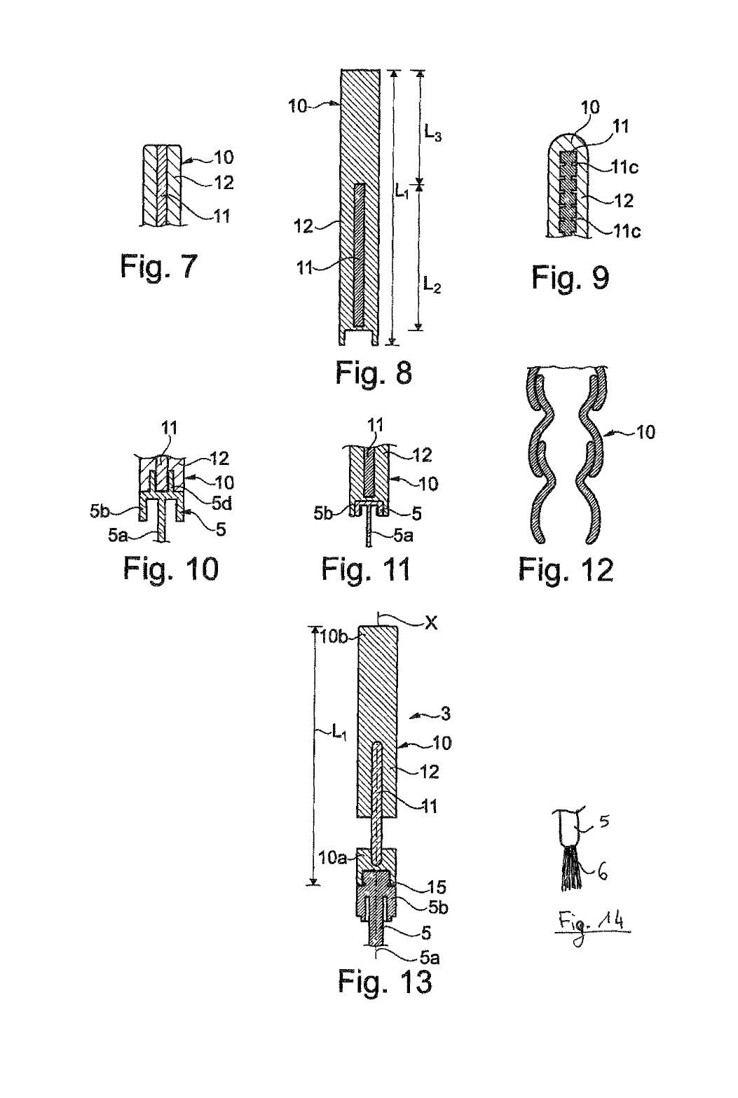

FIGS. 7 to 13 are schematic views, in partial longitudinal section, of variant embodiments, and

FIG. 14 is a detail view of one variant embodiment.

The packaging and application device 1 shown in FIGS. 1 to 4 has a container 2 containing the product to be applied and an applicator 3 which, when not being used to apply the product, can be mounted on the container 2 in order to close it, as illustrated in FIG. 1, and can be separated from the container in order to apply the product, as illustrated in FIG. 2. The applicator 3 extends along a longitudinal axis X. The product contained in the container is for example eyeliner.

The container 2 has for example a threaded neck onto which the applicator 3 can be screwed, the latter having a holder 5 that bears an applicator member 6 constituted for example by an applicator tip.

The applicator member 6 can be produced in various ways, in particular with a shape that is not rotationally symmetric, allowing the user to draw a more or less thick line depending on the orientation of the applicator member 6 with respect to the eyelid and the direction of movement thereon.

The applicator member 6 can be formed by a felt tip or by an end piece made of an elastomer material, which may or may not be flocked at its distal end. Examples of applicator members that can be suitable are described in particular in the publications FR 2 933 281, FR 2 947 702, FR 2 890 296 and U.S. Pat. No. 7,077,592.

In a variant, the applicator member may be a brush, as illustrated in FIG. 14.

The applicator 3 has a handle 10 defining a gripping surface that allows the user to hold the applicator while applying the product. The handle 10 is plastically deformable with shape memory at least along a part of its length.

The handle may extend for example in a rectilinear manner, as illustrated in FIG. 2. It may be curved as desired by the user, as illustrated in FIG. 4. The handle 10 may be deformed through 360°. The handle 10 may be configured such that, once it has been deformed to the maximum possible extent by the user, the inclination a of the proximal end 10b of the handle, of axis Y, with respect to the longitudinal axis X of the applicator when the latter has been made rectilinear is greater than 60°, or even greater than 90°, and less than 300°.

To this end, the handle 10 may have a core 11 made of a first, malleable material which is deformable with shape memory, in particular a metal material. The core 11 is covered with a sheath 12 of a second material. The sheath 12 is made of elastomer, for example.

The core 11 may be constituted by a wire of malleable metal such as copper, for example. In a variant, the core 11 is constituted by a succession of annular metal meshes that are friction crimped together in the manner of a metal hose as is encountered in some lamps, as illustrated in FIG. 12.

The handle 10 extends along a length L₁ which is around 90 mm in the example described. The handle has a greatest width l which is around 18 mm in the example described. This greatest width is situated in this case at the distal end 10a of the handle, closest to the applicator member 6.

The portion 10 that is deformable with shape memory extends for example along a length L₂ of around 80 mm.

The handle 10 may have a non-deformable portion that extends along a length L₃ of around 10 mm from the proximal end 10b.

Furthermore, the handle 10 may have at least two spaced-apart cross sections with different overall shapes.

The applicator may thus have a first cross section, taken perpendicularly to the longitudinal axis X of the applicator, with a circular overall shape, situated closer to the distal end 10a of the handle, that is to say close to the holder 5 and to the applicator member 6. The applicator may have a second cross section, taken perpendicularly to the longitudinal axis X, with a substantially lenticular overall shape, situated close to the proximal end 10b of the applicator.

The handle 10 may thus have two opposite facets 12b that are symmetric with respect to a median plane containing the longitudinal axis X of the application, when the latter is rectilinear.

The handle 10 may have snap-fastening ridges 15 on its inner surface, which are intended for fixing the holder 5 in the handle 10.

The holder 5 may be rigid. In the example illustrated, it has a stem 5a which supports the applicator member 6 at its distal end and an end piece 5b for fixing to the handle 10 at its proximal end, as can be seen in FIG. 3.

In order to use the device in FIGS. 1 to 4, the following steps can be successively implemented:

a) the handle 10 of the applicator is deformed in order to give it a given shape, for example the one illustrated in FIG. 4, and

b) the product is applied with the handle of the applicator in the shape it has been given.

The deformation given to the handle 10 may be different therefrom, being for example as illustrated in FIG. 5. The different shapes of the handle which can be obtained afford different ways of holding the applicator in the hand, as illustrated in FIGS. 6a and 6b by way of example.

In the exemplary embodiments which have just been described, the core 11 of the applicator extends along less than the entire length of the handle 10. If this is not the case, this does not represent a departure from the scope of the present invention.

By way of example, a variant embodiment in which the core 1 extends as far as the proximal end 10b of the handle 10 has been illustrated in FIG. 7.

In the variant embodiment in FIG. 8, the core 11 extends along a length L₂ that is slightly greater than half the total length L₁ of the handle 10. The non-deformable portion of the handle 10 extends along a length L₃ that is slightly less than half the length L₁ of the handle 10.

In the exemplary embodiment illustrated in FIG. 13, the core 11 extends along the entire length L₁, and is exposed along a portion of its length in which it is not covered by the sheath 12. The handle 10 is deformable only in that portion of the core which is not covered by the sheath 12, and the material of the sheath 12 is chosen so as to prevent the deformation of the core 11 and thus of the handle 10. Thus, the handle in FIG. 12 is deformable only along a portion of length L₂ that is much less than the length L₁.

The core 11 may have a constant cross section, or, as in FIG. 9, have annular grooves 11c that favour its deformation.

The holder 5 may also be fixed to the handle 10 in a different way from that described above. The holder 5 may for example have a collar 5d which is fitted on the core 11 of the handle 10, as illustrated in FIG. 10.

In the exemplary embodiments which have just been described, the end piece 5b for fixing the holder 5 is positioned in continuation of the surface of the handle 10 when it is in place on the latter. This does not have to be the case, and the fixing end piece 5b may for example be inserted entirely in a corresponding housing in the handle 10, as illustrated in FIG. 11.

Needless to say, the invention is not limited to the examples that have just been described. In particular, the features of the various examples illustrated can be combined within variants that are not illustrated.

The expression “having a” should be understood as being synonymous with “comprising at least one”, unless specified to the contrary.

1. Applicator for applying a cosmetic or care product, which extends along a longitudinal axis and has:
an applicator member,
a holder for the applicator member, and
a handle that defines a gripping surface allowing the user to hold the applicator during the application of the product, the handle being deformable with shape memory at least along a part of its length.

2. Applicator according to claim 1, wherein the handle extends initially without deformation in a rectilinear manner along the longitudinal axis of the applicator.

3. Applicator according to claim 1, wherein the handle extends along a length greater than 25 mm, better still greater than 30 or 40 mm.

4. Applicator according to claim 1, wherein the handle is deformable in a portion that is deformable with shape memory and extends along a length of between 20 and 100 mm, better still between 30 and 90 mm.

5. Applicator according to claim 1, wherein the handle has a non-deformable portion that is disposed at a proximal end thereof.

6. Applicator according to claim 1, wherein the handle has a core that is deformable with shape memory and is made of a first material, in particular a malleable metal material, and a sheath covering the core, made of a second material.

7. Applicator according to claim 1, wherein the holder is rigid.

8. Applicator according to claim 1, wherein the handle is configured such that it can be deformed through 360°.

9. Applicator according to claim 1, wherein the force to be applied by the user in order to deform the handle is between 0.5 and 10 N.

10. Applicator according to claim 1, wherein the handle is configured such that, once it has been taken to its maximum deformation by the user, the inclination of the proximal end of the handle with respect to the longitudinal axis of the applicator holder is greater than 360°.

11. Packaging and application device having a container containing a cosmetic or care product, in particular an eyeliner, to be applied, and an applicator according to claim 1.

12. Device according to claim 1, wherein the product is liquid.

13. Method for making up the eyelid, wherein use is made of an applicator or a device according to claim 1 to draw a line on the eyelid.

14. Makeup method, using an applicator or a device according to claim 1, wherein the following steps are implemented:
a) the handle of the applicator is deformed in order to give it a given shape, and
b) the product is applied with the handle of the applicator in the shape it has been given.
